(19) United States
(12) Patent Application Publication
(10) Pub. No.: US 2011/0246219 A1
(43) Pub. Date: Oct. 6, 2011

(54) System and Method of Integrated Logistics Management for Inventory

(76) Inventors: Gregory Smith, Ponte Vedra, FL (US); Stacy Smith, Orange Park, FL (US); Sebastian Rudbach, Atlantic Beach, FL (US)

(21) Appl. No.: 13/050,232

(22) Filed: Mar. 17, 2011

Related U.S. Application Data
(60) Provisional application No. 61/389,189, filed on Oct. 1, 2010.
(60) Provisional application No. 61/340,402, filed on Mar. 17, 2010.

(51) Int. Cl. G06Q 10/00; G06Q 50/00

(52) U.S. Cl. 705/2; 705/28

(57) ABSTRACT

A system and method of managing the logistics of trays and lots of medical inventory, such as surgical implants, from the manufacturer's initial release until final discharge by the end user healthcare provider. The system is driven by one or more software applications running on a central, secure server accessible by remote handheld telecommunication devices. The software platform comprises several assemblies integrated into the system, the assemblies including: a database access layer assembly, a business rules assembly, an entity or business object assembly, a web service assembly, a database connection assembly, a presentation assembly for a handheld device and a presentation assembly for a website and windows program. Additional features of the system could include a geographic map, an inventory list of trays and corresponding lots, an imaging assembly to read barcodes or other optic identifiers, an Food and Drug Administration news assembly incorporating a live RSS news feed for releases about product defects or recalls, a web browser, and a notebook assembly to enter customized notes in free form text.

This application claims the benefit of U.S. Provisional Patent Application Ser. No. 61/389,189, filed on Oct. 1, 2010, and of U.S. Provisional Patent Application Ser. No. 61/340,402, filed on Mar. 17, 2010.

### BACKGROUND

In the medical industry, surgical implant devices and the instruments for installing them are organized by the manufacturer into trays, and subcategorized into lots. The lots can be the surgical implant devices or the medical instruments needed to install such implants. Each tray is comprised of one or more implants and/or instruments, with each tray having a unique alphanumeric identifier for inventory purposes, and each lot having a unique lot number. Each tray has an arrangement of implants or instruments predetermined by the manufacturer.

Current smartphone applications tailored to track inventory are limited to tracking shipment information of each tray. The shipping information is accessed via the courier's website, and the smart phone application acts as a reporting tool showing the user the shipping status of the shipment. These applications offer limited information, such as the time the courier received the shipment, the estimated time and address of delivery, and in some cases the location of the package during the shipping process. Notably, these applications track shipments, as opposed to trays or lots.

Other inventory logistics systems are capable of managing inventory from a handheld device, but lack integrated features to track shipping information or data relating to use of inventory. Some such inventory systems are driven by RFID tags, which can create logistics discontinuities when the radio signal is disrupted, such as by a lack of electric power.

Additionally, the prior art does not teach a comprehensive logistics or tracking system in which one can track the location of a specific medical implant during the lifecycle of the device. Thus, it can often be difficult or impossible to locate defective devices that have been recalled by regulatory authorities, such as the United States Food and Drug Administration.

The present system seeks to overcome the limitations of previous systems by providing a comprehensive, integrated system for using a handheld telecommunication device to manage the logistics of medical device inventory throughout the lifecycle of the device.

### SUMMARY OF THE INVENTION

The present invention comprises a system and method of managing the logistics of trays and lots of surgical implants throughout the entire lifecycle of the device from the manufacturer's initial release until the instrument or implant is finally discharged by a medical service provider (i.e. disposing of an instrument or final expiration of the device). The system comprises software running on a central, secure server accessible by remote devices, such as handheld telecommunication devices or smart phones, including the iPhone®, Blackberry Storm™, or the like. The software platform comprises several assemblies integrated into the system, the assemblies including: a database access layer assembly, a business rules assembly, an entity or business object assembly, a web service assembly, a database connection assembly, a presentation assembly for a handheld mobile device and a presentation assembly for a website and windows program. The functionality of the system can comprise features such as a geographic map (i.e. Google® maps), an inventory list of trays and corresponding lots, an imaging assembly to read barcodes or other optic identifiers, an Food and Drug Administration (“FDA”) news feed assembly incorporating a live RSS feed for FDA releases about product defects or recalls, a web browser, and a notebook assembly to enter customized notes in free form text. The handheld device can access the software through a secure remote connection.

Users of the device are organizations that supply medical inventory, and in most cases each user will have multiple representatives, each requiring remote access to the system from a unique handheld. A user must contact the operator to obtain access credentials for the system. Upon initial activation of a user account with such credentials, the user must create a profile by entering data pertaining to the user's representatives and existing inventory.

During the life cycle of a tray, the tray and its corresponding lots enter into the software system upon shipment by the manufacturer, and the software tracks the location of the shipment to its delivery destination. When the shipment reaches its medical facility destination, a representative confirms the receipt location, thus logging the whereabouts of each tray by using the mapping assembly of the software. When a medical service provider discharges a lot, such as by implanting a device into a patient, the representative is on location to verify and log depletion of that particular lot by manually inputting appropriate data or by using the imaging assembly to scan the lot's optic identifier. Upon discharge of a lot, the software uses the inventory assembly to automatically send a message to the manufacturer to order corresponding replacement inventory.

By accessing the inventory assembly, the representative can obtain data about all inventory in the user's profile, the location of the inventory, and data about the inventory, such as dates, times, the name of medical facilities, mailing address of the location, and other customized data. The FDA assembly uses a live RSS to provide the representative with hourly FDA warnings or recalls for specific lots. Thus, the representative, who is typically in the operating room during implant procedures, can prevent medical procedures that use devices for which the FDA has revoked approval.

The user application software additionally comprises a web browser so that the representative is not required to exit the software application to access the web browser running separately on the handheld device. The notebook assembly allows the representative to enter free form notes for any reason.

### BRIEF DESCRIPTION OF THE DRAWINGS

FIG. 1 is a diagram showing the connectivity of the system's hardware components.

FIG. 2 is a diagram showing an alternate embodiment of the connectivity of the system's hardware components.

FIG. 3 is a diagram showing an alternate embodiment of the connectivity of the system's hardware components.

FIG. 4 is a diagram showing an alternate embodiment of the connectivity of the system's hardware components when a load balanced web farm architecture is used.

FIG. 5 shows one embodiment of the architecture of the website.

FIG. 6 shows a typical layout of the web application.

FIG. 7 is a diagram illustrating a typical web suite architecture used in the tracking system.

FIG. 8 is a diagram showing the interrelation of the application's functionality modules.

FIG. 9 is a flow chart showing the steps of operating the system for logistics management.

FIG. 10 is a diagram showing the work flow of the loaner program.

### DETAILED DESCRIPTION OF THE INVENTION

### I. Overview.

With reference to the drawings, the invention will now be described with regard for the best mode and the preferred embodiment. In general, the invention is a system for and method of providing integrated logistics management of inventory, wherein the system is operable from a single application on a mobile telecommunication device, such as a smart phone, tablet computer, or the like. The system is capable of providing real-time tracking and logistics management of inventory throughout the lifecycle of each tracked object. The system can be used to track inventory in a variety of industries, such as the medical, automotive, shipping, retail, or other industries. For the purposes of illustration, the system will be described herein in relation to logistics management of surgical implants. The embodiments disclosed herein are meant for illustration and not limitation of the invention. An ordinary practitioner will understand that it is possible to create many variations of the following embodiments without undue experimentation.

In general, medical inventory, such as surgical implant inventory, is categorized into trays, and subcategorized into lots. Each tray is issued a unique alphanumeric identifier, or tag, by the manufacturer, and each lot inside the tray is further identified by a unique lot number. The lots can be the surgical implant devices or the medical instruments needed to install such implants. As used herein, the term “inventory” or “medical inventory” means trays and their corresponding lots.

### II. Components and Connectivity.

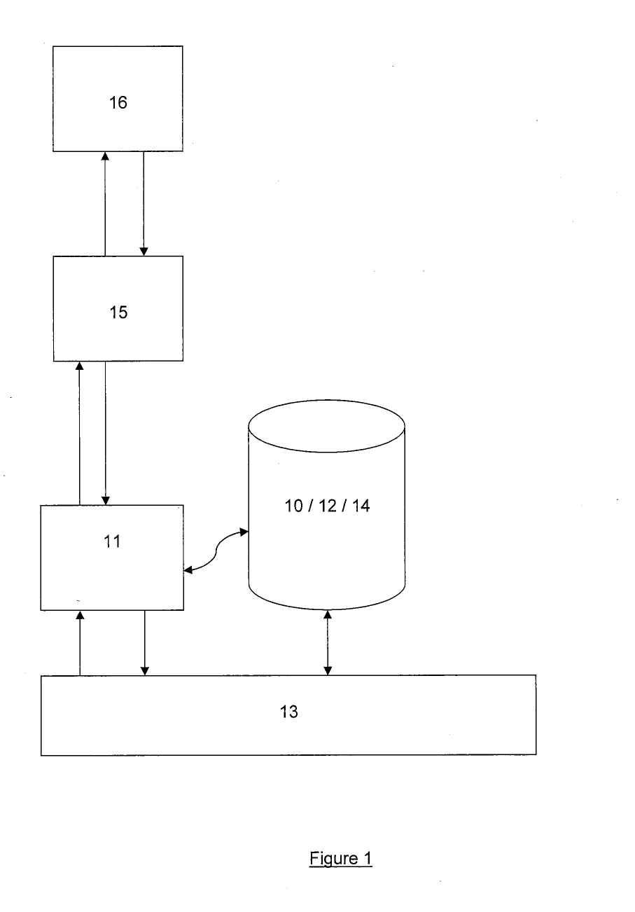

Referring to FIG. 1, the most basic embodiment of the logistics management system generally comprises the following basic components: a server 10, a communication service 11, a database 12, one or more assemblies 13 to facilitate communication between the database 12 and the communication service 11, a website 14, a consuming handheld mobile device 15 supporting a user software application 16 (shown in FIGS. 1-3). The server 10 could be a web server, such as an SQL web server that supports the website 14, and the website 14 can also communicate directly with the assembly 13. The communication service 11 is a means for communicating between the server and the device 15 or other devices, such as computers or other communication entities, such as a variety of web services. The communication service 11 could be an HTTP web request, an XML envelope, or TCP/IP protocol suite. The database 12 is a database that is accessible via the Internet. The assembly 13 is a library of compiled web services or other communication services 11. The user application 16 uses the communication service 11 to communicate with the database 12 and the assembly 13 via a commercially available network 17, such as a 3G network, broadband, the Internet, or other cellular or wireless network.

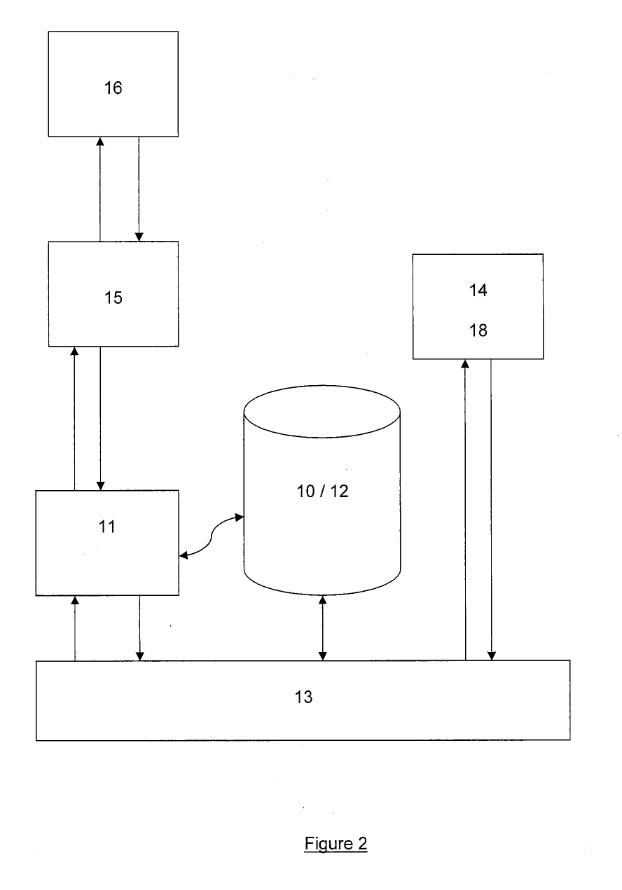

The server architecture of the system is a matter of design choice, and it may be advantageous in additional embodiments to provide one or more ancillary or redundant servers 18 to promote system reliability (see FIGS. 2-4). In one embodiment, the servers 10, 18 are secured with digital security that complies with the minimum digital security standards set forth in the Health Insurance Portability and Accountability Act (“HIPPA”). The communication service 11 is comprised of one or more application programming interfaces (“API”), many suitable forms of which are commercially available or can be created without undue experimentation. In FIGS. 1-3, the mobile device 15 can be a variety of handheld telecommunication devices, such as smart phones, like the iPhone®, Blackberry Storm™, tablet computers such as the iPad, Motorola Xoom, Google Honeycomb, or the like. The mobile device 15 comprises a GPS locator, as will be described in more detail below. The user software application 16 is a piece of software capable of being downloaded onto the mobile device 15 via the wireless network 17. The user software application 16 interfaces with the mobile device's 35 available features, such as the GPS geo-location feature, camera, video recorder, electronic map such as Google® maps, or other features. In another embodiment, the user application 16 can comprise a web browser so that the user does not have to exit the user application 16 to access the web browser on the mobile device 15. The user application 16 can also comprise a notebook so that the user can write customized notes about the inventory or surrounding conditions, and associate such notes with a specific item of inventory for later recall by the user.

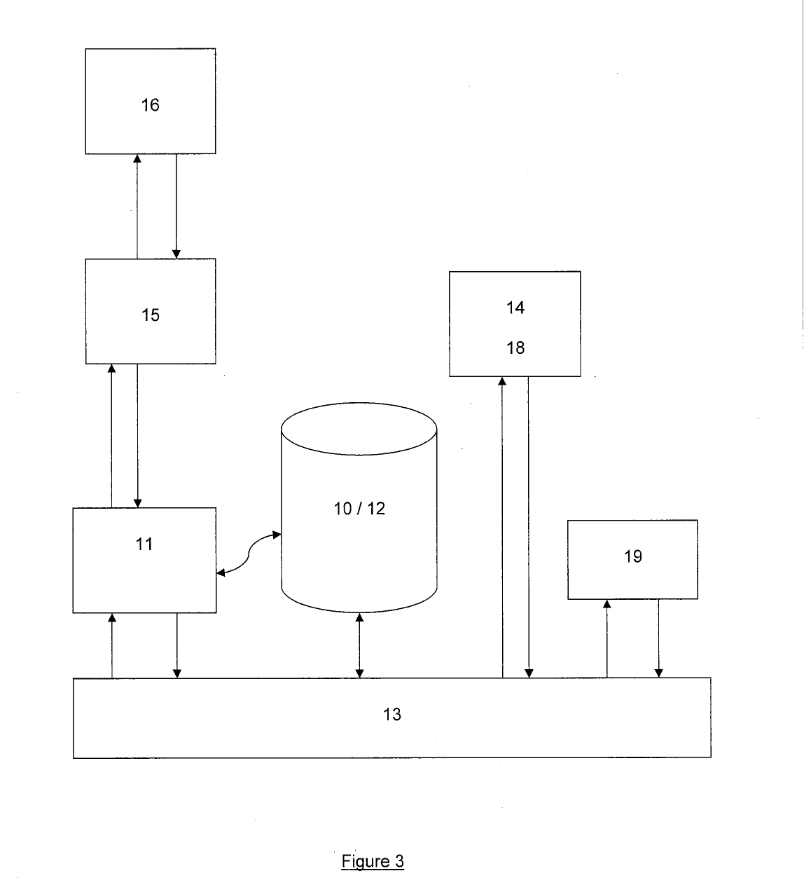

As shown in FIG. 3, another embodiment of the system can comprise a web application 19, such as a PC application or other application available via the world wide web, from which the user's administrator can manage the user's account and profile from a remote computing device, as described below.

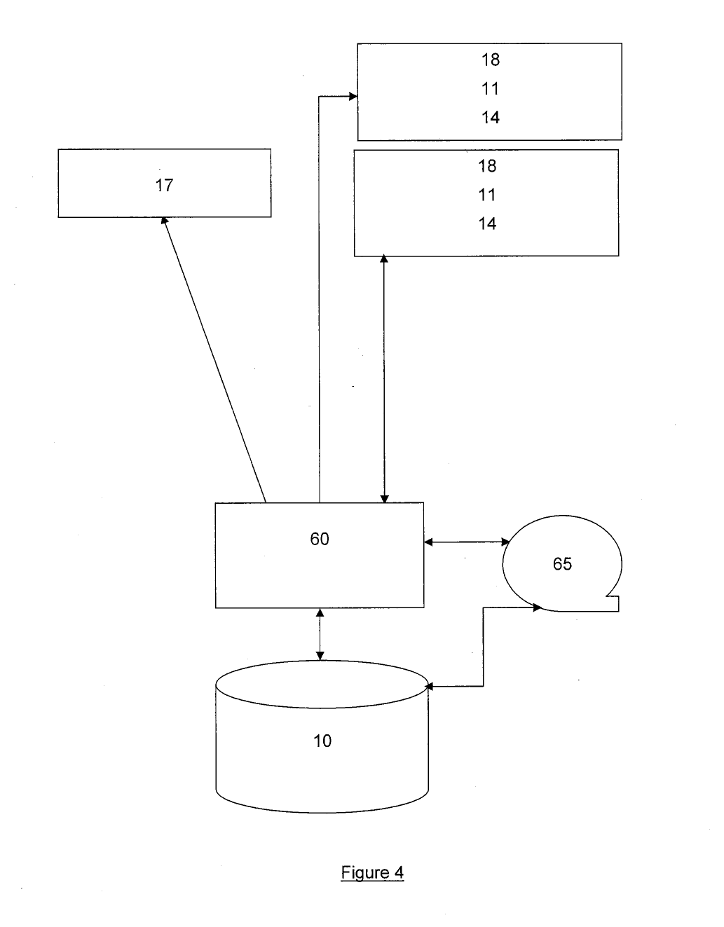

In another embodiment, shown in FIG. 4, the system comprises a load balanced web farm 60 supporting multiple servers 10, 45 in addition to support backup features 65, such as redundant servers and databases. The architecture of the web farm 60 is a matter of design choice and will vary by application. However, design of the web farm 60 design will not require undue experimentation of the tracking system.

### III. Software Application.

The system for and method of logistics management is driven by a software application comprised of several assemblies 13, which are libraries, or groupings of programming code, that fulfill the following roles for the system to function: a database access layer assembly, a business rules assembly, an entity or business object assembly, a web service assembly, a database connection assembly, a presentation assembly for a handheld device, and a presentation assembly for a website and windows program.

III.A. Assemblies.

(a) Database Access Layer Assembly.

This assembly abstracts from other assemblies 13 all activity related to the database 12, such as the activity of retrieving or saving data, and makes itself available for communication with other assemblies without being directly tied to them. This process is also known as encapsulation.

(b) Business Rules Assembly.

This assembly facilitates all related logical operations necessary to provide functionality to the system. For example, such functionality could include user account management, inventory management, execution of available features of the mobile device 15 or software applications, or other functions allowed by the system. This assembly also uses an encapsulation process and can be utilized in any direction in the logic flow without exposing anything internal to an external assembly 13 communicating with this assembly 13.

(c) Entity Object Assembly.

The entity object assembly provides a programming model based data schema. This assembly creates, manages, processes, and destroys all data that conceptually correlates to the real-space facts and information about items or objects of interest. For example, the entity object assembly could create, manage, process, or destroy data relating to trays, companies, delivery status parameters, location, or lifecycle of a tray or its associated lots.

(d) Web Service Assembly.

The web service assembly enables a device to process programming logic required for account validation, inventory management, and business rule processing by placing the applicable code assemblies on a remote server 10, 45. The mobile device 15 is then capable of invoking logic embedded in the remote assemblies 13 without having to house or store any of the programming logic on the device itself. The web service assembly can also be accessed by the associated user application 16 the same way.

(e) Database Connection Assembly.

The database connection assembly 13 encapsulates all required logic to access the inventory, account, and user database by providing a mechanism that enables the communication service 11, when invoked, to access the database connection assembly 13 and, in turn, retrieve the requested data and pass it back through the communication service 11 to the device 15 or user application 16.

(f) Presentation Module Assembly.

The presentation module assembly related to the mobile device 15 is all programming logic that is required to be installed on the mobile device 15 itself, which includes rendering logic for buttons, tables, text, and the navigation features permitting uninhibited navigation through the user application 16.

(g) Presentation Assembly.

The presentation assembly applies to the assemblies 13 and programming code responsible for the display of data, buttons, text and tables on the associated user application 16.

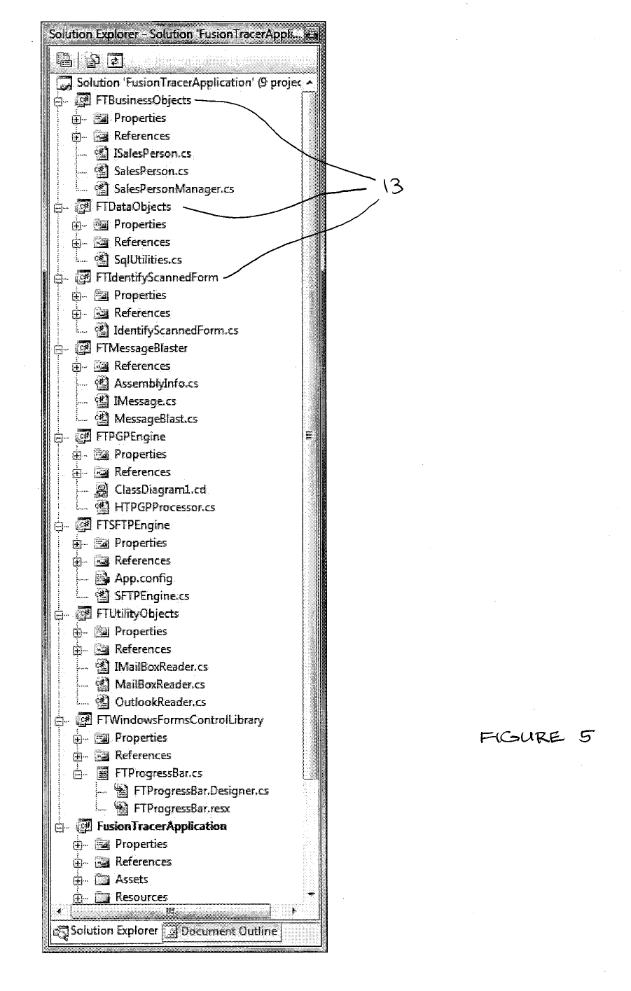

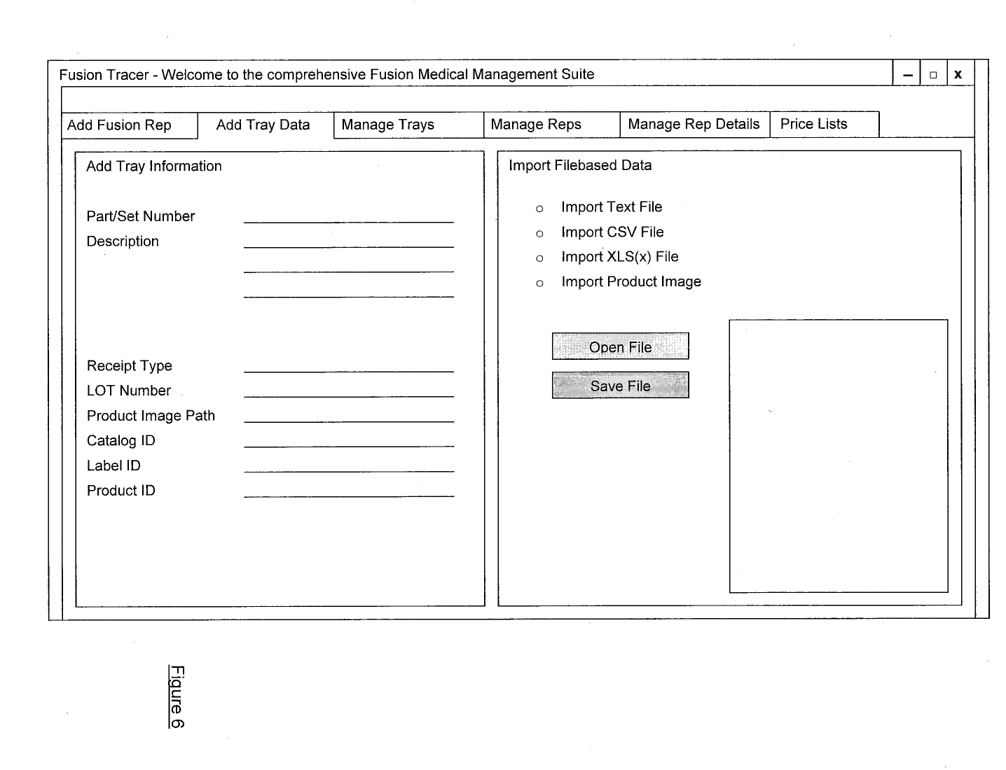

By way of further examples, FIG. 5 shows a typical architecture of the website 14, illustrating the interrelation between typical libraries, or assemblies 13. FIG. 6 shows the typical layout and architecture of the web application 19, which would typically be operated by the user's administrator. The web application 19 includes features for managing sales teams and trays, such as by adding and managing representatives, adding and managing tray data, updating price lists. The web application 19 may also comprise fields for entering data, such as tray or lot information or other fields. The mobile device 15 uses the communication service 11 to communicate with the server 10. These features can be comprised within a web suite 30, as described below.

### IV. Web Suite

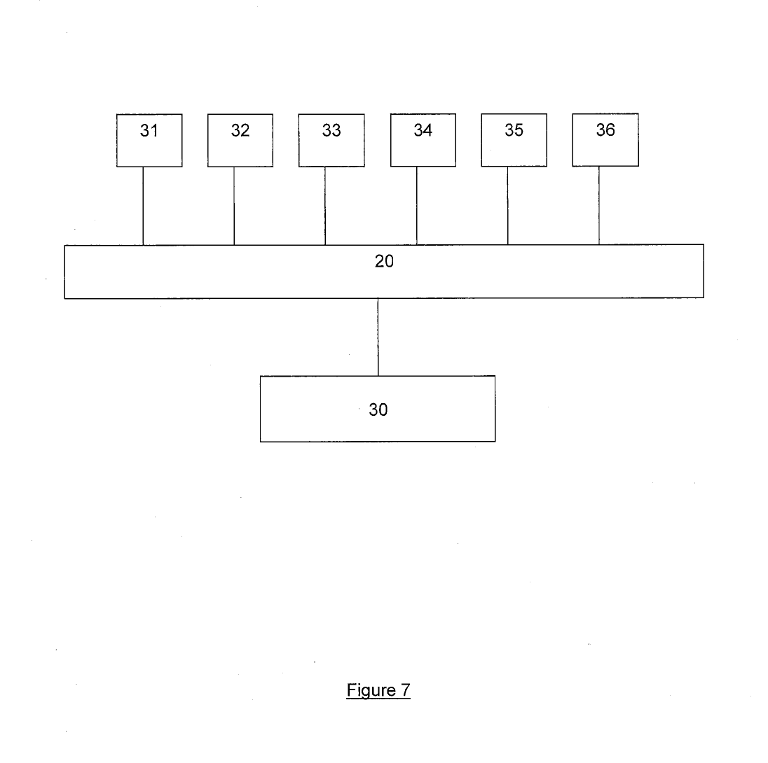

Referring to FIG. 7, in one embodiment, the system comprises a web suite 30 that forms the interface between the user and the system, enabling the user to manage the flow of process of the system and interact with data stores. The features in the web suite 30 can be added or adjusted to accommodate a variety of users, needs, or applications of the tracking system. The user can access all such data stored in the user's profile, which enables the user to have a centralized management system for all of its representatives, facilities, providers, and patients. In typical embodiments, the web suite 30 will comprise features such as a dashboard 31, scheduler 32, case viewer 33, revenue tracker 34, inventory manager 35, and doctor preferences 36, to name a few. The dashboard 31 is the graphical interface between the user and the system, and the dashboard 31 displays visual indicators for access to features and processes. For example, the user uses the dashboard 31 to access the system during the user authentication process and to set up the user account and profile.

The scheduler 32 permits the user to send and receive scheduling data and information to and from the database 12. The user can input, retrieve, or edit information relating to schedules for inventory delivery, cases, representatives, or other such events, people, and processes. The case viewer 33 permits the user to access all information relating to a particular case, such information including data relating to the representative, facility, provider, patient, date of the case, the inventory to be used, and other data relevant to the particular application of the tracking system. The case viewer 33 allows the user to have access to all cases within the user's profile, and to display certain data during a certain time period. For example, the user can retrieve and view all revenue received from facility X on day Y. A wide variety of other reporting parameters can also be selected.

The revenue tracker 34 permits the user to access, review, and store all electronic documentation and data relating to payments for and revenue from the inventory used in the cases. For example, such electronic documents may include electronic versions of purchase orders, requisitions, receipts, financial documents, or the like. Revenue data can include sales price, discounts, rebates, financial and lending data, or other such information. The revenue data can be sorted by representative, facility, case, doctor, specific inventory item, or the like. Revenue data can be tracked and reported for specific time intervals in connection with other data selection criteria. For example, the user can use the revenue tracker 34 to retrieve and view the revenue generated by representative X during month Y.

In one embodiment, the revenue tracker 34 can be configured to interface with the user's accounting software so that the user's accounting books are automatically updated with revenue information.

The inventory manager 35 enables the user to manage all inventory included within the user's profile. For example, the user can access, review, and edit data relating to one or more representatives, the inventory currently at one facility, inventory allocated to one or more cases, and all data about the inventory, such as the manufacturer, the delivery date, expiration date, quantity, contents, and other such pertinent information. In one embodiment, the user can perform a “radius search,” which will return to the user information about all inventory within a specified radius of a certain geographic location. Thus, the user can readily identify all inventory within a certain facility or town.

The doctor's preferences 36 feature allows the user to access, edit, and view information relating to preferences by certain doctors with whom the user's representatives typically interact. For example, the user may discover through the doctor's preferences 36 feature that a certain doctor prefers cases to happen on certain weekdays, or that he or she prefers one brand of implant device over other brands. Any such unique preferences by the doctor can be recorded by the representative and stored in the system on the database 12 for future reference by the administrator or other representatives.

The web suite 30 can comprise additional features or functionality, depending on the needs of the user or other determinative considerations.

### V. Application Functionality and Platform.

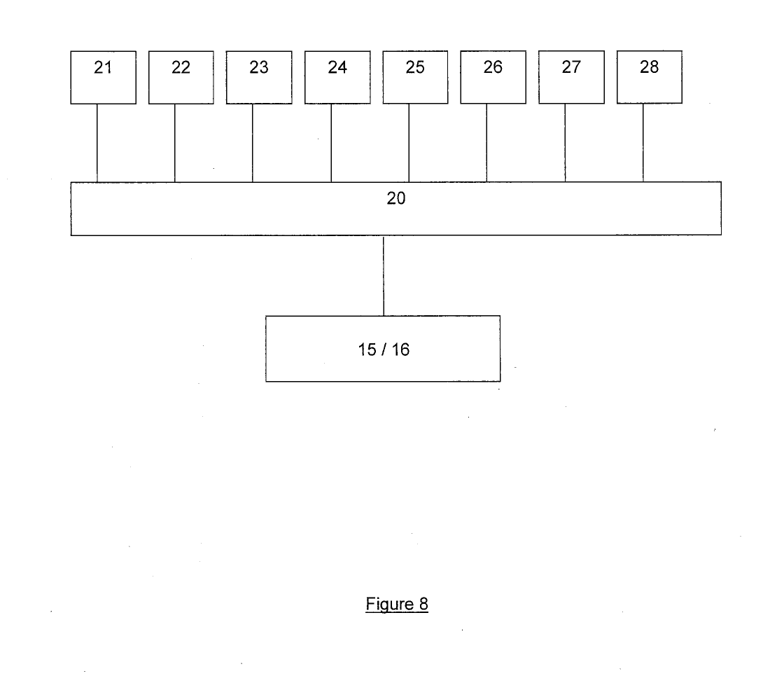

Referring to FIG. 8, the application 16 comprises a platform 20 incorporating multiple functionality modules that can be selected and arranged as appropriate for the particular purposes of the tracking system. An exemplary list of modules include a receiver 21, pin locator 22, inventory viewer 23, case scheduler 24, case manager 25, optical scanner 26, communicator 27, and preferences reporter 28, among others.

The receiver 21 is an onboard inventory input utility in the application 16 on the mobile device 15 that is used as a graphical interface for data entry into the database 12, as well as data retrieval from the database 12. The representative may input into or retrieve from the database any data relating to inventory, specific trays or lots, biologistics, delivery information, quantity, expiration date, geographic or facility data, or any other stored data relating to the user, profile, inventory, doctor, patient, facility, or the like. The representative can also use the receiver 21 to feed lots into a tray, if so required, by capturing part number, lot number, part description, expiration date, and quantity within the context of the distributor. This allows the distributor to add unforeseen or newly discovered inventory into the profile aside from possibly receiving EDI data inventory transfers into the distributor's profile. In addition, the receiver 21 allows for immediate GPS based location pinning of the entered data at the location where the input occurs, as described below.

The pin locator 22 is a module used to pinpoint a geographic location. For example, the pin locator 22 can pinpoint the geographic location of the mobile device 15, and therefore the representative. Additionally, the pin locator 22 can be used to “pin” the geographic location of inventory by using the GPS locator in the mobile device 15. The inventory is “pinned” when the user uses the pin locator 22 to record and store the geographic data associated with the current geographic location of the inventory. The pin locator 22 can be configured to report geographical locations in a variety of forms, such as street addresses, the geographic coordinate system (i.e. latitude and longitude), or other forms. For example, the latitude and longitude readings returned from the GPS feature in the mobile device 15 can be converted to a street address and displayed on an electronic or online map, such as Google® maps. As the physical location of the inventory changes with time, the user should update the inventory's recorded location by re-pinning the inventory as needed. Later, when the whereabouts of the inventory is needed, the user can use the inventory viewer 23 module to determine the last pinned location of the inventory, as described below.

The inventory viewer 23 module permits the user to view the current inventory in the user's profile, including a view of the current tray and lot numbers and any other parameters stored in the database 12 in association with the inventory. Quantity, status, availability, and location may be such parameters, if so desired by the user and stored in the database 12. The inventory viewer 23 can also permit the user to edit the inventory listings, such as by deleting mobile device 15 entered data, entering notes pertaining to inventory data, or editing other parameters.

In one embodiment, the inventory viewer 23 provides immediate search and reporting capability of inventory by location, radius in miles and name of the device, implant or tray. In another embodiment, the inventory viewer 23 provides satellite and street level views of currently located inventory rendering parts and lots that are potentially part of a tray, device, or implant. In another embodiment of the inventory viewer 23, the main screen also provides for searching not only the tray, device, or implant by name, but also allows the user or distributor the option to perform an FDA regulation-based search by actual part or lot numbers, and the inventory viewer 23 displays the location of any inventory found.

In another embodiment, the pin locator 23 also provides a tracking means, which is a means for tracking the location as a function of time for each piece of inventory throughout its entire lifecycle. For instruments, the lifecycle begins at the time of the initial shipment by the manufacturer until the time that the instrument lot is depleted, such as by completing a surgery or final disposal of the instrument lot or tray by a healthcare provider. For implant devices, the lifecycle begins at the time of the initial shipment by the manufacturer and continues until the time that the device expires, which may be several days, weeks, or years after the device is implanted into the patient. When a tray or lot is depleted from the inventory, such as by discarding the lot or implanting it into a patient, the representative updates the corresponding inventory data, and the system will automatically notify the manufacturer that a replacement tray or lot is needed and the location of where such inventory is needed. The tracking means includes the functions of identifying, logging, and recording a tray number or a lot number, and recording the time and location of the tray or lot during the entire lifecycle. In another embodiment, the tracking means comprises tracking parameters carrying additional information about the inventory, such as the surgery location, physician name, tray identification, a picture of the facility, or other information as desired. The system enables the tracking function by connecting to the database 12, through the same communication service 11 that the mobile device 15 is remotely hydrating and utilizing.

In another embodiment of the system, the inventory viewer 23 further comprises a reporting function that provides a web based view for users or representatives to access the tray, lot, surgery data, or other tracking parameters in real-time as soon as data has been entered. This view, which is always associated with unique login credentials (e.g., for the representative), provides for a map view, a data view and detailed breakdown view pertaining to the data being logged on the remote handheld device. Such data could include location (GPS) data, detailed tray information, surgery data, or other tracking parameters the representative has entered. The system enables the reporting function by connecting to the database 12, through the same communication service 11, that the mobile device 15 is remotely hydrating and utilizing.

The case scheduler 24 permits the user to schedule surgery procedures, or “cases,” in which a tray or lot will be used or depleted. In the case scheduler 24, the user can input or edit parameters relating to each case 29, such parameters including the name and location of the health care facility, nature of the procedure, the doctor's name, relevant patient information, the date, time, and room number of the procedure, and any other relevant or desired information. The case scheduler 24 also permits the user to modify or edit the parameters as needed or desired.

The case manager 25 is a module used by the representative during a surgical procedure to control inventory levels, purge used lots, and create requisitions for depleted inventory. Thus, the case manager 25 gives the user immediate purge control over all inventory used in a case, deleting any depleted stock out of the system in real-time. It also provides for an electronic requisition system for replenishing medical inventory that has been depleted from a predefined profile of medical inventory. In one embodiment, for example, the requisition system captures all parts, lots, prices, and descriptions onto a template or custom requisition form that can be submitted to a manufacturer allowing for immediate replenishment of tray or device lots.

The case manager 25 module displays input form fields and inventory data corresponding to a particular case. The case manager 25 provides a control interface for manipulation of the medical inventory data stored in the database 12 corresponding to a specified medical procedure. For example, the representative uses the case manager 25 to enter information for inventory used during a surgery, or to recall or edit any other information related to the particular case. The system then, through a method called in the web service assembly, stores in the database the tracking parameters pertaining to the case-specific tray or lot, as identified by the tray or lot number. This storage procedure, or precompiled structured query language statement, facilitates the normalized distribution of the data in the data tables responsible for saving tray, surgery, lot data, tray data, or other tracking parameters or case information.

The representative can also use the case manager 25 to create requisitions. A requisition is a re-order form to replenish exhausted or depleted inventory. For example, when a lot is used in a surgery, the inventory of that lot is typically replenished by completing a form, or a requisition, requiring a signature authorizing the manufacturer to resupply the inventory identified in the document. One embodiment of the case manager 25 comprises functionality in which the requisition is automatically prepared for signature when the representative designates inventory as exhausted, depleted, or expired in the course of a case. When such inventory is removed from the representative's inventory listing, the case manager 25 automatically hydrates an electronic requisition form with the data necessary to reorder such depleted inventory. The representative, doctor, or other authorizing party can then use the touch-activated screen on the device 15 to sign the requisition form via a “signature capture.” The signature capture allows the user to use his or her finger to trace his or her signature onto the touch screen. The graphics capability in the touch screen framework capture a line-form digital image of the signature in a common format, such a JPEG, TIFF, bitmap, or the like. The signature capture then conversts the signature image to a binary code capable of being transmitted over the Internet as a data file. This data is transmitted to the server 10 where the data is reassemble on the server 10 and inserted into the signature line of a document. For example, the reassembled signature could be inserted into the signature line of the “requisition image” of the completed and fully hydrated requisition form permanently tagged with the image of the requisition form with the image of the signature. The requisition image is then made available for access via the Internet by the device 15, web suite 30, a web browser, or other means. This process will appear as a real-time action to the signatory. The case manager 25 can also email a copy of the requisition image to the representative for subsequent review and processing. The final requisition form can also be electronically transferred to the representative for distribution to the healthcare facility or manufacturer.

The optical scanner 26 module comprises an imaging assembly capable of reading and logging data contained in an optic or electronic identifier, such as a bar code or an RFID tag. The optical scanner 26 module serves as a backup option to identify inventory. In many cases, the manufacturer will affix a barcode to the inventory as a means of optical identification. The barcode can contain unique identifying information about the tray or other inventory. In one embodiment of the present system, the system incorporates a mobile device 15 that comprises a camera capable of capturing the barcode image, and software within the mobile device 15 will process the image to extract the numerical value of the barcode, thereby identifying the inventory. In more advanced embodiments of this system, the camera and mobile device 15 are capable of capturing and processing multiple barcodes in one image.

In another embodiment of the present system, the optical scanner 26 comprises bluetooth laser reader, which is detached from the mobile device 15, but offers the user the most generic barcode reading capability in the industry. The laser reader permits the user to decide which entry field within the receiver or case manager is targeted by the bluetooth laser. This, in turn, gives the user the ability to decide if a part or lot or other barcode is within the context of the selected field.

The communicator 27 module that provides a chat client available to the user and representatives registered to the user's profile. Thus, the communicator 27 provides an “intra-company” or “intra-profile” communication forum having features such as SMS text message options, thread-discussion forums searchable by topic, and a stored data log that retains all communications in a database. These and other such features of the communicator 27 can be selected and arranged as desired for a particular purpose without falling outside the scope of the present system.

The preferences reporter 28 is a knowledge base available within the mobile device 15. The preferences reporter 28 is capable of receiving, storing, and recalling on-demand information relating to the physician's name, place of business, affiliation with healthcare providers, preferred items of inventory, preferred style and manner of surgery preparation, or the like. The information stored in the preferences reporter 28 can be retrieved and displayed in a scrollable format.

In another embodiment, the application 16 comprises a news feed that receives real-time industry based news releases, such as releases from regulatory agencies. For example, the news feed is capable of receiving news releases from the Food and Drug Administration regarding warnings, alerts, or recalls corresponding to certain medical inventory.

These and other modules can be selected and arranged as needed to suit certain user profiles or applications of the inventory tracking system, which is not limited to any particular selection or arrangement of these modules. For example, the system may comprise a built-in shipping tracker, enabling the distributor to enter any shipping tracking number to automatically receive the shipping status and information.

### VI. Method of Use.

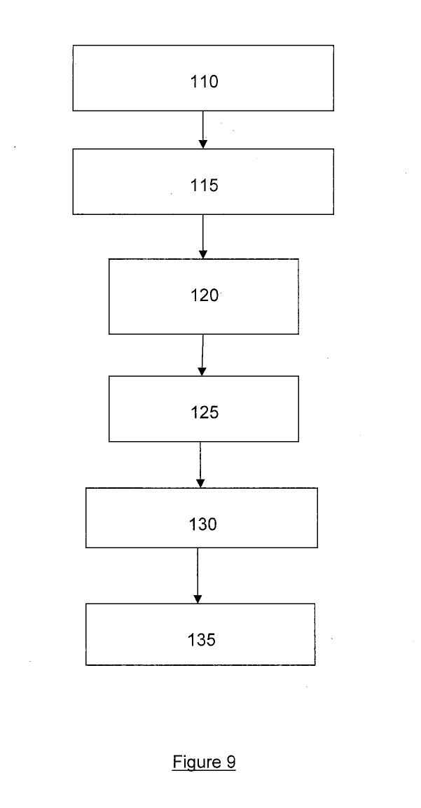

In use, the logistics management system functions as a single, integrated application accessible from the mobile device 15 via the Internet. Users of the system are medical device suppliers, each one having multiple sales representatives. The system is provided by the management system operator. Referring to FIG. 9, the operator provides the service to use the system through the steps of issuing user credentials 110, establishing a user account 115, customizing a portal configuration 120, customizing a profile configuration 125, establishing representatives 130, and enabling system operation 135.

In the step of issuing user credentials 110, the operator generates user credentials for the purposes of user authentication. Each user is assigned a unique set of credentials, which could include alphanumeric characters or digital signatures in the form of passwords, user names, or other identifying information. The user must use the credentials to access the system via the website 14. In the step of establishing a user account 115, the operator collects from the user certain identifying information, such as entity name, address, industry, a description of the user's inventory, the desired level of service that the user would like, and any relevant payment information. In the step of customizing a portal configuration 120, the operator provides the user the opportunity to customize the portal for the website 14 according to the user's inventory, representatives, unique business structure, desired level of service, and any other factors considered by the user.

In the step of customizing a profile configuration 125, the operator provides the user the opportunity to customize the user profile according to the user's inventory, representatives, unique business structure, desired level of service, and any other factors considered by the user. In addition, the user can establish one or more account administrators. The user profile can be organized according to geographic zones, sales locations, healthcare providers (i.e. hospitals or physician practices), representatives, trays, lots, or a variety of other parameters identified by the user. The information submitted by the user is communicated through the website 14 to the database 12 where it is used to populate the user's profile. After the initial setup, inventory or representatives can be added or removed from the user's profile by either the user or the operator by modifying a flag on the user's data record. In one embodiment, modifying this flag only removes it from view but is not erased from the database 12. In another embodiment, data can be erased only with consent of the operator, thereby reducing instances of fraud, malicious intent, or similar perceived threats.

The data storage within the database 12 table schema follows data normalization, where inventory, user, and other account data are segregated into separate tables and associated through primary and foreign keys. In one embodiment, user data is communicated via a strict data processing protocol ensuring safety, security, and protection through applying PGP, SHA-1, and MD5 encryption standards that are applied to the processing of any user related data. For example, in one embodiment, the data storage occurs on industry proven database platforms on digitally and physically secured servers. The database servers 10, 45 also follow disaster recovery, redundancy, and availability standards that are accepted and approved by certain industries handling sensitive data.

In the step of establishing representatives 130, the administrator designates one or more sales representatives, each having a unique mobile device 15. To activate the representatives, each representative can use the mobile device 15 to retrieve their assigned access and account information from the administrator. In one embodiment, this is accomplished by providing logic in the system that invokes a standard port email transfer of the generated access information to the email provided to the system during the user's setup. In another embodiment, the automated email transfer includes an embedded pass code used by the mobile device 15. The representative can then use the pass code to access the system and download the user application 16 onto the mobile device 15. After initial access of the system, the representative can change the auto-generated pass code to an alphanumeric or digital code unknown to the operator.

The application 16 is then used to establish a connection to the web service assembly in order to form a connection between the mobile device 15 and the tracking system. The application 16 provides the representative access to the features and functionality associated with each of the modules described above. For example, the application 16 will present process indicators for the receiver 21, pin locator 22, inventory viewer 23, case scheduler 24, case manager 25, optical scanner 26, communicator 27, and preferences reporter 28 modules, among others. Through these indicators, the representative can access the application 16 and system functions, data, and features available through each respective module.

By way of example, the representative can use the inventory viewer 23 module to retrieve inventory data for display on the device 15. The inventory data retrieved and displayed by the device 15 is based on the account setup, in particular pertaining to location and zone in which the representative is active. The inventory data, geographic data, and all other data pertinent to the representative are downloaded onto the mobile device 15 as a locally cached file, thus permitting availability of the data in the event of a loss of system connectivity. This file is automatically refreshed to synchronize with the user's inventory data on the database 12, and the automatic refresh rate can be set to a predetermined time interval, the login of the representative, the restoration of lost connectivity, or a number of other intervals or triggering events. The file could also be automatically refreshed upon changes to inventory data occurring either on the database 12 or in the locally cached file.

In the step of enabling system operation 135, the operator provides the representative with the opportunity to establish geographic coordinates of the health care provider, the representative, the trays, or other pertinent information.

In one embodiment of the method of logistics management, the method further comprises providing an RSS feed assembly and functionality. The RSS feed assembly is a live, real-time data and information feed reporting news and releases issued from the FDA or other sources, and the feed is accessible by the user application 16 on the mobile device 15. By accessing and reviewing the feed, the representative can remain appraised of an changes in FDA approval relating to the inventory managed by the representative. For example, the FDA news feeds can be electronically gleaned for news relating to specific tray or lot numbers, and these numbers can be compared to and synched with the inventory data in the user's profile. Upon locating an inventory number in the database 12 for which an FDA release has been issued, the inventory in the database 12 can be flagged to obtain the representative's attention. After seeing a flag associated with a specific tray or lot number, the representative will know that an FDA release has been issued, and the release should be reviewed before using the corresponding inventory in a medical procedure. When a case is in progress, the FDA flags can be communicated real-time via the case manager 25, scheduler 32, or inventory manager 35 so that either the representative, the administrator, or both, are alerted to the FDA warning. Such real-time alerts during a case can allow the representative to prevent a recalled device from being used in a procedure or implanted into a patient as a result of poor information flow.

In one embodiment of the method of logistics management, the method further comprises the step of providing functionality or assemblies that enable the user to pinpoint the representative's geographic location through the handheld devices built-in GPS geo-location. When the healthcare facility receives a shipment of inventory from a manufacturer, the representative is on location to receive the shipment and pin the location of the trays and lots via the pin locator 22 module. The user then accompanies the shipment to its physical storage location, such as a storage room or an operating room. The representative is also present in the operating room during the surgery to record the tray and lot numbers of any inventory depleted during the surgery. Thus, the built in geo-location features in the representative's mobile device 15 serve as an electronic tag to locate the inventory at any given time after delivery to the healthcare facility. The administrator can set the location of inventory by invoking logic that retrieves the current coordinates and other relevant data, and saving the data to the database 12 tables via the communication service 11. This is done by accessing the mobile device's 15 location framework and utilizing its exposed longitude and latitude values and hydrating them into the responsible business object that is embedded in the presentation assembly. To facilitate this functionality, the user application 16 can comprise an electronic mapping feature, such as Google® maps, enabling the representative or the user to see the geographic location of the representative, and therefore the inventory.

In another embodiment of the method of logistics management, the method additionally comprises the step of making user's stored GPS coordinates in the database available to the mobile device when the user requests tray inventory assigned to him. A custom view then facilitates the reverse geo-location logic embedded in the presentation assembly of the device to process the previously saved coordinates and show, in map view, the location represented by the coordinates saved. This gives the user a record of the exact location of a tray and its current state of storage or use.

In another embodiment of the method of logistics management system, the method additionally comprises the step of enabling the user to manually manipulate the state or status of a tray related to its availability prior, during, and after surgery.

In the inventory tracking system, the user's trays or lots are automatically entered into the user's profile upon shipment by the manufacturer. While the inventory is being shipped, its location is tracked by gathering and reporting the shipping information available from the courier, most of which report tracking information on various websites. Upon delivery of the inventory to the healthcare facility, a representative is on location using the mobile device 15 to receive the shipment and log the relevant tracking parameters into the user's profile. As the inventory is stored or maintained at the location of the healthcare facility, the representative uses the mobile device 15 to log any notable changes in the inventory's tracking parameters. The representative is then on location for the surgery where specific lots are depleted via installation into a patient, and the representative uses the mobile device 15 to log this depletion of the specific lot. Thus, after the inventory is delivered to the healthcare provider, the representative and his or her mobile device 15 is at the location of the inventory. In this manner, the physical location of the inventory can be pinned via the GPS location features of the mobile device 15. Additional tracking parameters of the inventory are also available from the database.

For example, if the FDA issues a health warning or recall of a specific lot, the representative can immediately identify the location of each such lot currently in inventory, in addition to the tracking parameters of each such depleted lot. In embodiments where the representative receives FDA notice real-time, the representative has the ability to notify healthcare providers during a surgical procedure that the lot used in the procedure carries a newly discovered health risk or other defect. In addition, by using the tracking parameters for each depleted lot, the representative can identify the patient in which each lot was installed, the healthcare provider that performed the procedure, and the manufacturer that made the lot. Thus, each manufacturer, healthcare provider, and patient can minimize health risks or take preventative action by being warned of the danger communicated in the FDA notice or recall. Individual patients can be notified that they have received a device that poses certain risks, according to the FDA notice. Otherwise, after the conclusion of a surgery, and hence usage of a tray have concluded, a next surgery is scheduled for the tray, and the representative will then use the mobile device 15 to revise coordinates and enter lot data, tracking parameters, and associated surgery information into the system for real-time retrieval via the mobile device 15 or the website 14.

### VII. Loaner Program

VII.A. Overview

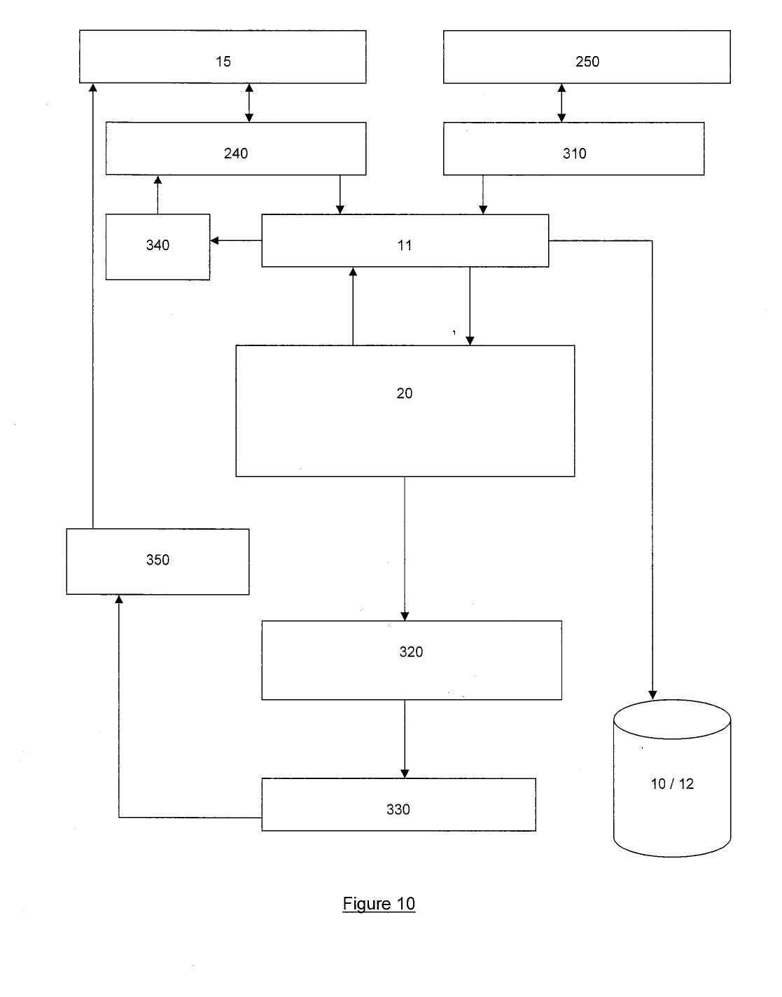

Referring to FIG. 10, the tracking system can comprise an optional feature permitting sales representatives to loan certain inventory. This “loaner program” permits the representative to use inventory that is infrequently used or too cost prohibitive to purchase. To support the loaner program, the tracking system is modified to allow the manufacturer to create a user profile. The distributor then designates in its own profile one or more manufacturers with which the distributor would like to be associated. These designated manufacturers are available to the distributor to receive “loaner requests” submitted by the sales representatives of either the distributor or the manufacturer.

A loaner request 240 comprises a list, or designated set, of generalized tray types. A set of specific loaner tray serial numbers may be referred to as a “unit” or “kit.” Units may be built using one of two assembly methods depending on the manufacturer: (1) Ad-hoc and (2) Kit Definition. The ad-hoc method is the method by which a set of trays is bundled to form a kit without regard to the tray's individual serial number. Generally, ad-hoc is where a family of tray types is bundled at the time of allocating trays to form the “unit.” The kit definition method is the method by which specific tray serial numbers are always bundled collectively in order to form the “unit.” In this model, special unit number characteristics are stored on each tray record in the tracking system to be able to track all trays belonging to a single unit. At the time of tray allocation, the allocation manager can view this unit characteristic to ensure that only trays of the same unit are being allocated.

Generally, the representatives use the case scheduler 24 to initiate any needed loaner requests 240 at the time of scheduling a case. The representative uses the mobile device 15 to place orders and view status of the loaner request 240. The representative originates a loaner request 240, which goes into a queue that is only readable and manageable on the web suite 30.

VII.B. Loaner Request Lifecycle

The life-cycle of a loaner request 240 consists of four stages designated 340 by four indications: (1) unfilled, (2) filled, (3) shipped and (4) cancelled. When the loaner request 240 is initially saved, it enters the system under the default status of “unfilled.” After the loaner request 240 has been “hard allocated” it goes into a status of “filled.” When the unit is shipped by the manufacturer, the loaner request 240 moves to a status of “shipped” and is affixed a tracking number 350. A representative may choose at anytime in the life-cycle other than “shipped” to cancel the loaner request 240. A cancelled loaner request 240 releases any hard allocations associated with it. Optionally, the indication of “shipped” trays represents the inventory going out while the indication of “en route” trays represents those coming in.

VII.C. Tray Allocation Process

The manual tray allocation process starts with an allocation manager 250 selecting a loaner request 240 from the unfilled queue 310. Based on the selection of a loaner request 240, a list of requested tray types in the loaner request 240 is automatically obtained. The manager 250 compares 320 this list of tray types against the inventory database for all tray numbers belonging to the tray types identified. The tray status, or availability, is then indicated by a visual indicator, such as a light, icon, distinct font, or the like. The manager 250 then “hard-allocates” available trays to the loaner request 240. When a tray type is unavailable, the manager 250 sends an “unavailable” indication to the sales representative, via the sales representative's mobile device 15, that submitted the loaner request 240, and the representative can either select an alternate tray type, cancel the loaner request 240, or remove the unavailable tray from the loaner request 240, allowing the remainder of the loaner request 240 to be filled. When the manager 250 finishes allocating trays to a loaner request 240, the loaner request 240 is marked as “filled” in order to remove it from the unfilled queue.

The allocation process can be automated by an intelligent suggestion module in which the system automatically retrieves a subset of tray serial numbers that will be allocated to the selected loan types designated in a certain loaner request 240. The allocation manager 250 invokes the suggestion module electronically, such as by clicking a button labeled “suggested trays.” Upon activation, the suggestion module parses the manufacturer's inventory listings to locate the best match criteria for the tray types listed in the loaner request 240, and the module automatically returns suggest trays based on predesignated match criteria. The allocation manager 250 can then choose to accept the suggestion, or override the module to make a manual selection.

The intelligent suggestion modules pulls trays based on the following hierarchy: (1) tray status, (2) tray usage, and (3) padding. The framework for this module is very flexible and allows for the addition and accommodation of new metrics in the future.

The embodiments disclosed above are merely representative of the system and method and not meant for the purposes of limitation. One having ordinary skill in the art would understand that the individual features of several disclosed embodiments are interchangeable with the features of other embodiments. For example, the system could comprise additional assemblies or any combination of the assemblies disclosed herein, as desired. Also, multiple formations of the system architecture are a matter of design choice. Consequently, it is understood that equivalents and substitutions for certain elements and components set forth above are part of the invention, and the true scope of the invention is set forth in the claims below.

We claim:

1. A system for tracking medical inventory, said system comprising:
a server supporting a database populated with data corresponding to medical inventory, said server connected to a mobile device via a communication service, said mobile device having a GPS locator; and
an application supported by the mobile device, said application having a receiver for interfacing with the database by transmitting data between the mobile device and the database, a pin locator for correlating the geographic location data from the GPS locator with specified medical inventory for the purpose of pinning the geographic location of the medical inventory without a tracking device being physically affixed to the medical inventory, and an inventory viewer providing an interface for viewing the data transmitted by the receiver.

2. The system of claim 1, wherein the application further comprises a real-time news feed that receives news releases related to the medical industry, said news releases delivering warnings, alerts, and recalls corresponding to specified medical inventory.

3. The system of claim 1, wherein the application further comprises a case manager that provides a control interface for manipulation of the medical inventory data stored in the database, said medical inventory data corresponding to a specified medical procedure.

4. The system of claim 3, wherein the case manager further comprises a requisition system for replenishing into a predefined data profile data corresponding to medical inventory that has been depleted during a medical procedure.

5. The system of claim 4, wherein the mobile device further comprises a touch screen, and the requisition system further comprises a signature capture that captures from the touch screen a line-form digital image of the signature, converts said image to a binary code data file capable of being transmitted over and electronic network, transmits the binary code data file to the server where the data file is reassemble on the server and inserted into a signature line of an image of a document.

6. The system of claim 2, wherein the application further comprises a case manager that provides a control interface for manipulation of the medical inventory data stored in the database, said medical inventory data corresponding to a specified medical procedure.

7. The system of claim 6, wherein the case manager further comprises a requisition system for replenishing into a predefined data profile data corresponding to medical inventory that has been depleted during a medical procedure.

8. The system of claim 7, wherein the mobile device further comprises a touch screen, and the requisition system further comprises a signature capture that captures from the touch screen a line-form digital image of the signature, converts said image to a binary code data file capable of being transmitted over and electronic network, transmits the binary code data file to the server where the data file is reassemble on the server and inserted into a signature line of an image of a document.

9. A system for tracking medical inventory, said system comprising:
a server supporting a database populated with data corresponding to medical inventory, said server connected to a mobile device via a communication service, said mobile device having a GPS locator;
an application supported by the mobile device, said application having a receiver for interfacing with the database by transmitting data between the mobile device and the database, a pin locator for correlating the geographic location data from the GPS locator with specified medical inventory for the purpose of pinning the geographic location of the medical inventory without a tracking device being physically affixed to the medical inventory, and an inventory viewer providing an interface for viewing the data transmitted by the receiver;
a web application providing a management interface by which data and parameters corresponding to a predefined user profile can be manipulated from a remote computing device; and
a real-time news feed that receives news releases related to the medical industry, said news releases including warnings and recalls relating to specified medical inventory.

10. The system of claim 9, wherein the application further comprises a case manager that provides a control interface for manipulation of the medical inventory data stored in the database, said medical inventory data corresponding to a specified medical procedure.

11. The system of claim 10, wherein the case manager further comprises a requisition system for replenishing into a predefined data profile data corresponding to medical inventory that has been depleted during a medical procedure.

12. The system of claim 11, wherein the mobile device further comprises a touch screen, and the requisition system further comprises a signature capture that captures from the touch screen a line-form digital image of the signature, converts said image to a binary code data file capable of being transmitted over and electronic network, transmits the binary code data file to the server where the data file is reassemble on the server and inserted into a signature line of an image of a document.
